(19) United States
(12) Patent Application Publication
(10) Pub. No.: US 2024/0228950 A1
(43) Pub. Date: Jul. 11, 2024

(54) MICROPATTERNED 3D HYDROGEL MICROARRAY IN FLUIDIC CHANNELS FOR SPHEROID-IN-GEL CULTURE

(71) Applicant: NANYANG TECHNOLOGICAL UNIVERSITY, Singapore (SG)

(72) Inventors: Han Wei HOU, Singapore (SG); Chengxun SU, Singapore (SG); Yon Jin CHUAH, Singapore (SG)

(21) Appl. No.: 18/558,687

(22) PCT Filed: Apr. 28, 2022
(86) PCT No.: PCT/SG2022/050263

§ 371 Date: Nov. 2, 2023

(30) Foreign Application Priority Data
May 3, 2021 (SG) 10202104559S

(51) Int. Cl. C12N 5/00; C12M 1/12; C12N 5/071; C12N 5/09

(52) U.S. Cl. CPC C12N 5/0062; C12M 25/02; C12N 5/0688; C12N 5/069; C12N 5/0693; C12N 5/0697; C12N 2513/00; C12N 2533/54; C12N 2537/10

(57) ABSTRACT

Herein disclosed is a method of encapsulating a spheroid in a gel, the method comprising: providing a frame comprising a base and an island protruding from the base; depositing one or more suspensions on the island, wherein the one or more suspensions comprise different cells; arranging the frame to have the one or more suspensions hang from the island in a direction which gravity acts to render growth of the spheroid; depositing a gel on the spheroid with the spheroid resting on the island to have the gel encapsulate the spheroid; and arranging the frame against a substrate with the base distally positioned from the substrate (i) to have the gel confined between the island and the substrate and (ii) to have the gel encapsulate the spheroid. Disclosed herein includes a device configured to render a spheroid encapsulated in a gel, the device comprising: a frame comprising a base and an island protruding from the base; and a substrate, wherein the frame is arrangeable against the substrate with the base distally positioned from the substrate (i) to have the gel confined between the island and the substrate and (ii) to have the gel encapsulate the spheroid.

### CROSS-REFERENCE TO RELATED APPLICATION

This application claims the benefit of priority of Singapore Patent Application No. 10202104559S, filed 3 May 2021, the content of it being hereby incorporated by reference in its entirety for all purposes.

### TECHNICAL FIELD

The present disclosure relates to a method of encapsulating a spheroid in a gel. The present disclosure also relates to a device configured to render a spheroid encapsulated in a gel.

### BACKGROUND

The overall success rate for phase I to phase III clinical trials is estimated to be 13.8%, and may be as low as 3.4% for oncology medication. This translational failure may be largely due to the reliance on in vivo animal testing and the lack of preclinical in vitro model that can accurately predict the drug responses in humans, such as lack of in vitro tumor model that may predict the drug efficacy and toxicity in a physiologically relevant context. Conventional two dimensional (2D) cultures on tissue culture flasks and three dimensional (3D) transwell co-culture tend not to mimic tumor microenvironment. 3D spheroids may be more physiological, but are cultured in suspension without presence of extracellular matrix (ECM) or co-culture with vasculature. These factors may affect spheroid viability, drug diffusion kinetics and IC50.

With advances in tissue engineering and microfluidics, more complex in vitro 3D cellular models, including spheroid cultures and organ-on-a-chip platforms, have been developed and increasingly used in recent years. Such complex 3D models may still utilize spheroids to offer the higher biological complexity in terms of structural and functional properties by recapitulating the cell-cell interaction and tissue-like architecture. The 3D spheroids may be traditionally cultured in suspension (e.g. the hanging drop method or round bottom 96-well plate), and lack a surrounding extracellular matrix (ECM), which may plays a significant role in mediating instructive signals for cell polarization, retention, and mobilization.

On the other hand, microengineered organ-on-a-chip systems may have been widely used to reconstitute key functional unit of human organs by precisely manipulating the fluid flow and control of 3D tissue structure and ECM microenvironments.

In any cell culture platforms, the incorporation of ECM/hydrogel patterning may be considered to establish (1) more physiological 2D cell monolayers on hydrogel surfaces, (2) 3D cell cultures using cell-laden hydrogels, and (3) co-cultures of multiple cell types by hydrogel compartmentalization to recreate complex 2D/3D tissue architecture. Classical surface tension-based hydrogel patterning in microfluidics may include the use of micropillars, or narrow openings. However, the intermittent physical barriers may give rise to discontinuous cell-ECM interface, which may hamper cell-cell and cell-ECM communication, or subjects cells to differential biochemical and biophysical cues.

To address these issues, several studies may have been carried out on hydrogel patterning techniques to form continuous cell-ECM interface in enclosed microchannels using a phaseguide, recoverable elastic barrier, or suspended gel. While a spheroid-in-gel culture may be achieved by patterning a spheroid-containing ECM using the above methods, they are limited by the inability to manipulate single spheroids and precisely control spheroid positions within the ECM in enclosed microchannels. These tends to lead to the need to adapt hydrogel patterning techniques on open chambers to accommodate single spheroid assays. However, as spheroid formation tends to be performed in cell suspension, this requires manual transferring of individual pre-formed spheroids into microfluidic devices for hydrogel encapsulation, which is laborious and prone to human error.

There is thus a need to provide for a solution that addresses one or more of the limitations mentioned above. The solution should at least provide for a spheroid-in-gel culture platform that allows for the precise positioning of single spheroids and integrates spheroid formation and in-gel culture on a single device.

### SUMMARY

Herein discloses a versatile method to pattern hydrogel in a geometrically defined microarray. A biomedical application of this method is exemplified in the examples section by the creation of a spheroid-in-gel culture platform that can include the following advantages: (1) tunable droplet size for optimal spheroid formation by hanging drop culture, (2) in-place encapsulation of spheroid with geometrically defined hydrogel pattern, (3) scalable for high content drug screening applications, and (4) feasibility to perform co-culture of spheroid with vascular cells to generate a vascular network surrounding the spheroid ECM region.

In a first aspect, there is provided for a method of encapsulating a spheroid in a gel, the method comprising:
providing a frame comprising a base and an island protruding from the base;
depositing one or more suspensions on the island, wherein the one or more suspensions comprise different cells;
arranging the frame to have the one or more suspensions hang from the island in a direction which gravity acts to render growth of the spheroid;
depositing a gel on the spheroid with the spheroid resting on the island to have the gel encapsulate the spheroid; and
arranging the frame against a substrate with the base distally positioned from the substrate (i) to have the gel confined between the island and the substrate and (ii) to have the gel encapsulate the spheroid.

In another aspect, there is provided for a device configured to render a spheroid encapsulated in a gel, the device comprising:
a frame comprising a base and an island protruding from the base; and
a substrate,
wherein the frame is arrangeable against the substrate with the base distally positioned from the substrate (i) to have the gel confined between the island and the substrate and (ii) to have the gel encapsulate the spheroid.

### BRIEF DESCRIPTION OF THE DRAWINGS

The drawings are not necessarily to scale, emphasis instead generally being placed upon illustrating the principles of the present disclosure. In the following description, various embodiments of the present disclosure are described with reference to the following drawings, in which:

FIG. 1A is a schematic diagram illustrating the workflow of CBV-based press-on hydrogel confinement method of the present disclosure.

FIG. 1B is a schematic illustration of the two-step photolithography and polydimethylsiloxane (PDMS) replica molding for device fabrication.

FIG. 1C is a demonstration of gel loading and press-on gel confinement using collagen I (3 mg/ml) mixed with red or green food dye. Cross-sectional image of the device (bottom panel). Red region (darker region) indicates the position of gel after press-on gel confinement.

FIG. 2 shows bright field images of different shapes of hydrogel island before hydrogel patterning (top). Collagen I (3 mg/ml, containing 10 mM FITC) was confined within different shapes of hydrogel island under the CBV effect (bottom). Scale bar=1 mm.

FIG. 3A shows collagen I (3 mg/ml, containing 10 mM FITC) confined within circular islands of different diameter. Scale bar=1 mm.

FIG. 3B shows that different edge distance between two adjacent circular islands patterned with Collagen I (3 mg/ml, containing 10 mM FITC). Scale bar=1 mm.

FIG. 4 shows hydrogel (containing 10 mM FITC) with different crosslinking mechanisms confined within the patterned circular island. Scale bar=1 mm.

FIG. 5 shows a schematic of workflow for on-chip spheroid-in-gel formation.

FIG. 6A shows fluorescence images of FITC-containing droplets.

FIG. 6B shows droplet height across different diameter of circular islands and volume of droplets on both hydrophobic and hydrophilic surfaces.

FIG. 6C shows effect of cell number, droplet volumes and island size on MCF-7 spheroid formation.

FIG. 7A shows a 5×6 microarray chip for spheroid-in-gel culture, specifically an image of water droplet (mixed with food dye) confined within the chip.

FIG. 7B is a bright field image of MCF-7 spheroid in hanging drop after culturing on chip for 2 days.

FIG. 7C is a cross-sectional view of the chip of FIG. 7A.

FIG. 8A shows bright field images of various size of MCF-7 spheroid in hanging drop.

FIG. 8B is a plot of evaporation rate of media droplet in three different environments.

FIG. 8C shows bright field image of an entire channel with 6 spheroids embedded within hydrogel. The distances between each island are not to scale.

FIG. 9 demonstrates for viability of spheroids after hydrogel encapsulation and culturing in different media (from day 4) on day 7. Calcein-AM stains for live cells (green) while ethidium homodimer-1 stains for dead cells (red). Scale bar=200 μm.

FIG. 10A demonstrates co-culture of MCF-7 spheroids with HUVEC in collagen and Matrigel. MCF-7 was labelled with DiO, F-actin—red, dapi—blue.

FIG. 10B demonstrates retrieval of spheroid-laden hydrogel using forceps and resuspension in a microtube. Spheroid (small white dots) can be observed with naked eyes and were highlighted with the arrows.

FIG. 11 shows comparison of spheroid area measured by F-actin signal (red—grey shaded region) and Hoechst signal (Blue—grey shaded portion). White dashed circle indicates channel boundary. Spheroids were embedded in Collagen I (3 mg/mL).

FIG. 12A is a schematic illustration of stepped height-based hydrogel patterning in a single lane hydrogel chip. Overlaid fluorescence and bright field image of the chip loaded with FITC-laden Collagen I (3 mg/mL) in the hydrogel channel (top). Cross-sectional view of the chip, white double-headed arrow indicates channel height of 170 μm, while the other yellow arrow (also a double-headed arrow) indicates channel height of 140 μm (bottom).

FIG. 12B are fluorescence images showing the gel loading process at different time points for Collagen I (1, 3 mg/mL, FITC-laden) and Matrigel (4, 8 mg/mL, FITC-laden). Yellow dotted line indicates the channel boundary.

FIG. 12C is a plot of loading speed of 1×PBS, Collagen I (1, 2, and 3 mg/mL), Matrigel (2, 4, 6, and 8 mg/mL) in the one-lane hydrogel chip.

FIG. 12D is a schematic illustrating the concept of multi-lane hydrogel confinement using a single stepped height for odd number of lanes, and two stepped heights for even number of lanes. Black (darker) arrows indicate the first-layer channels (lower height), red (lighter shade of grey) arrows indicate the second-layer channels (intermediate height), and blue arrows indicate the third-layer channels (highest height).

FIG. 12E is a schematic of sequential three-lane hydrogel loading.

FIG. 12F shows overlaid fluorescence and bright field image of the chip loaded with FITC-laden Collagen I (3 mg/mL) in the first and third lanes, and R6G-laden Collagen I in the second lane (middle). Cross-sectional view of the chip, white arrow indicates channel height of 145 μm, while yellow arrows indicate channel height of 120 μm (bottom).

FIG. 12G shows fluorescence image of the chip containing two lanes of HLF-laden Collagen I with a cell-free Collagen I in between. (F-actin-red, Hoechst-blue).

FIG. 12H shows the fabrication method for the 1-lane hydrogel chip, 3-lane hydrogel chip, and press-on hydrogel microarray chip.

FIG. 12I shows image of the 1-lane hydrogel chip (left) and 3-lane hydrogel chip (right).

FIG. 13 shows schematic of different surface tension-based hydrogel patterning techniques in enclosed microchannels. Leftmost image denotes traditional pillar-based method, center image denotes traditional phaseguide-based method, and rightmost image denotes stepped-height based method.

FIG. 14A is a schematic illustration of press-on hydrogel confinement on patterned islands on PDMS substrate.

FIG. 14B is a demonstration of gel loading and press-on gel confinement using Collagen I (3 mg/mL) mixed with red or green food dye (top). Cross-sectional image of the device, yellow arrows indicate the stepped height of ˜190 μm (bottom).

FIG. 14C shows fluorescence images of FITC-laden Collagen I confined within different shapes of hydrogel island. White dotted line indicates the shape of the patterned hydrogel island.

FIG. 14D is a schematic illustration of workflow for on-chip spheroid-in-gel formation.

FIG. 15A shows fluorescence images of FITC-containing droplets on hydrophobic and hydrophilic extruded circular island features.

FIG. 15B are plots of droplet height with different island diameter and volume of droplets on both hydrophobic (left plot) and hydrophilic (right plot) surfaces.

FIG. 15C demonstrates effect of cell number, droplet volumes and island size on MCF-7 spheroid formation.

FIG. 16 are plots of contact angle with different island diameter and volume of droplets on both hydrophobic (left plot) and hydrophilic (right plot) surfaces. Data was presented as mean±SD (n=3).

FIG. 17A shows image of water droplet (mixed with food dye) confined within a 5×6 microarray chip.

FIG. 17B shows stitched bright field image of spheroids in hanging drop after culturing on chip for 2 days.

FIG. 17C shows evaporation rate of media droplet in three different environments. Data are presented as the mean±SD (n=3).

FIG. 17D shows stitched bright field image of a single channel of the 5×6 microarray chip with spheroids embedded within collagen gel. Red circle indicates the region of hydrogel island, and blue circle indicates the region of the spheroid microwell.

FIG. 18 shows a cross-sectional view of one island on the microarray chip for spheroid-in-gel culture.

FIG. 19 shows FITC/FITC-10 kDa Dextran (0.1 μM) uptake into MCF-7 spheroid co-cultured with HUVEC in Collagen I (3 mg/mL) and Matrigel (4 mg/mL). After 24 hr incubation, channels were washed and fixed in 4% PFA for imaging. White dashed circle indicates the channel boundary.

FIG. 20 demonstrates viability of spheroids after hydrogel encapsulation and culturing in different media (from day 4) on day 7 (Calcein-AM—green, PI—red).

FIG. 21A shows co-culture of MCF-7 spheroids with endothelial cells (HUVEC) in collagen and Matrigel. MCF-7 was labelled with DiO, F-actin-red, and Hoechst-blue.

FIG. 21B shows merged fluorescent images illustrating viability of spheroid after PTX treatment (Calcein-AM-green and PI-red).

FIG. 21C is a plot of normalized fluorescence intensity of Calcein-AM for spheroids with or without HUVEC co-culture after 3 days of treatment with PTX 1, 100, and 500 nM. A PFA-fixed spheroid was used as negative control. Data are presented as the mean±SD (n=3).

FIG. 21D shows merged fluorescent images illustrating viability of spheroids and HUVEC in co-culture after 3 days of PTX treatment. (Calcein-AM—green and PI—red).

FIG. 22 is a reconstructed 3D fluorescence image of HUVEC layer surrounding the ECM region (Collagen I, 3 mg/mL) on one island of the chip (F-actin—red, Hoechst—blue).

FIG. 23 is a fluorescence image of endothelial cells cultured in the spheroid-in-gel chip (VE-Cad—green, Hoechst—blue, F-actin—red).

FIG. 24 is a plot of normalized fluorescence intensity of PI for spheroids with or without HUVEC co-culture after 3 days of treatment with PTX 1 nM, 100 nM, 500 nM. PFA-fixed spheroid was used as negative control. Data was presented as mean±SD (n=3).

FIG. 25 is a plot of normalized fluorescence intensity within the hydrogel island for untreated control, 100 nM and 500 nM Paclitaxel (PTX) treated HUVEC. FITC-dextran 70 kDa (10 μg/mL) was loaded into the chip and allowed to diffuse into the island for 60 min before images were taken and analyzed. The fluorescence intensity was expressed as fold change (time 60 min over 0 min), and normalized to the mean of untreated control. Results were as expressed as mean±SD, ** p<0.005.

FIG. 26 shows retrieval of spheroid-laden hydrogel using forceps and resuspension in a microtube. Spheroid (small white dots) can be observed with naked eyes and are highlighted with red arrows.

FIG. 27 demonstrates for stability of Collagen I (3 mg/mL) and Matrigel (4 mg/mL) at various flow rate. Hydrogel were mixed with fluorescent microbeads for visualization. Images were taken after 5 min of perfusion.

FIG. 28A is a schematic illustration of sequential hydrogel patterning in the dual-lane curved channel chip.

FIG. 28B shows a fabrication method using the 3-step photolithography and PDMS replica molding.

FIG. 28C is a schematic illustration of perfusion culture using the dual-lane curved channel chip.

FIG. 29 is a photo of the dual-lane curved channel chip loaded with Collagen I (top view). Cross-sectional view of the chip (bottom). Yellow arrows indicate the stepped height of ˜50 μm; red arrows indicate the stepped height of ˜100 μm.

FIG. 30A shows results of the one-inlet loading method. Collagen I (3 mg/mL) was loaded into the chip from one inlet and stopped immediately when overflowed. Green boxes (top row—4th and 5th images counting from left) indicate successful gel patterning; red boxes indicate failed gel patterning.

FIG. 30B shows results of the two-inlet-loading method. Collagen I (3 mg/mL) was loaded into the chip from one inlet to fill half of the channel, following which the other half was loaded from the other inlet. Gel loading was stopped immediately when overflowed. Green boxes (bottom row—2^{nd} to last image counting from left) indicate successful gel patterning; red boxes indicate failed gel patterning.

FIG. 31A is a schematic of gel traveling distance (arc length) calculation method.

FIG. 31B is a plot of the quantification of gel traveling distance (arc length) for chip 3/1/1 and 4/1/1. Results were expressed as mean±SD.

FIG. 32 is a gel loading results for chips with through hole lumen. Chips were loaded with Collagen I (3 mg/mL) for both hydrogel channels using the two-inlet loading method. Green boxes indicate successful gel patterning (2nd to 4th images counting from left); red boxes indicate failed gel patterning.

FIG. 33A is a photo of the chip loaded with Collagen I (3 mg/mL) before perfusion, rods circled in red are inlet seals.

FIG. 33B is a photo of the chip under perfusion with colored water (mixed with red dye) at 10 ml/min.

FIG. 33C is a photo of the chip after perfusion for 5 min at 10 ml/min.

### DETAILED DESCRIPTION

The following detailed description refers to the accompanying drawings that show, by way of illustration, specific details and embodiments in which the present disclosure may be practised.

Features that are described in the context of an embodiment may correspondingly be applicable to the same or similar features in the other embodiments. Features that are described in the context of an embodiment may correspondingly be applicable to the other embodiments, even if not explicitly described in these other embodiments. Furthermore, additions and/or combinations and/or alternatives as described for a feature in the context of an embodiment may correspondingly be applicable to the same or similar feature in the other embodiments.

The present disclosure relates to a method of encapsulating a spheroid in a gel and a device configured to render the spheroid encapsulated in the gel. Details of various embodiments of the present method and device, and advantages associated with the various embodiments are now described below. Where the embodiments and/or advantages have been described in the examples section further hereinbelow, they shall not be reiterated for brevity.

The method comprises providing a frame comprising a base and an island protruding from the base, depositing one or more suspensions on the island, wherein the one or more suspensions comprise different cells, arranging the frame to have the one or more suspensions hang from the island in a direction which gravity acts to render growth of the spheroid, depositing a gel on the spheroid with the spheroid resting on the island to have the gel encapsulate the spheroid, and arranging the frame against a substrate with the base distally positioned from the substrate (i) to have the gel confined between the island and the substrate and (ii) to have the gel encapsulate the spheroid.

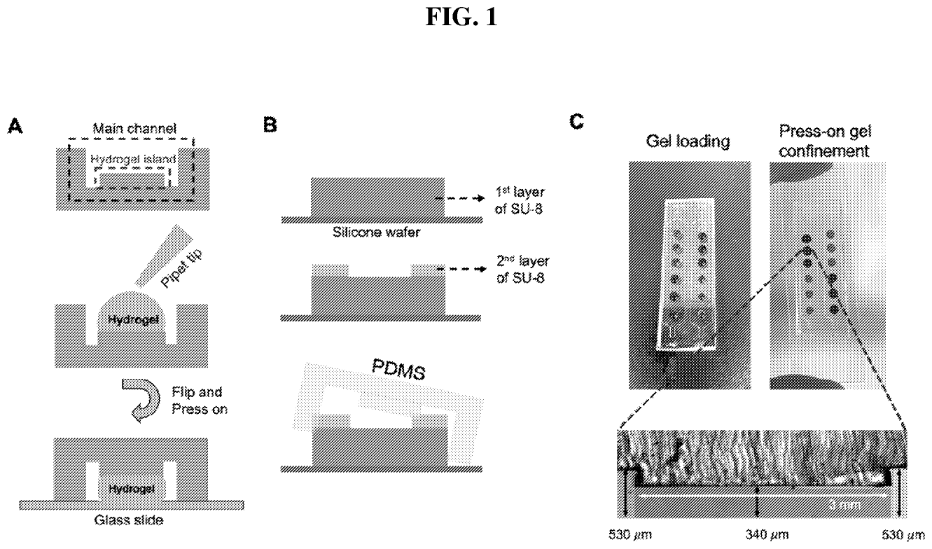

The term “frame” refers to a component of the device, and is herein exchangeably referred to as a “chip”. The frame, as configured and when arranged against the substrate, helps to confine the gel to a space, defined by the island and the substrate, via a capillary burst valve (CBV) effect, which FIG. 1A illustrates to provide a better understanding.

In the context of the present disclosure, the term “spheroid” refers to a three-dimensional (3D) cell culture, wherein cells aggregate during proliferation to form a sphere-like formation. The sphere-like formation may be a perfect sphere or has a shape that is substantially spherical (i.e. not a perfect sphere).

In various embodiments, the method may further comprise introducing one or more culture media to the gel after the gel encapsulates the spheroid. The one or more culture media may be introduced into the gel that has encapsulated the spheroid by injecting the one or more culture media into the gel.

In various embodiments, depositing the one or more suspensions may comprise mixing the one or more suspensions with the gel prior to depositing the one or more suspensions. Each of the one or more suspensions may contain a different type of cell. In other words, the method includes co-culturing of different cell types using the device and encapsulating the different cell types in the gel. Non-limiting examples of the cells include human umbilical vein endothelial cells, human lung fibroblasts, human breast cancer cells, or a mixture thereof.

In various embodiments, the one or more suspensions may comprise or may be deposited at a volume of at least 1 μL, at least 2 μL, at least 3 μL, at least 4 μL, at least 5 μL, etc.

In various embodiments, the method may further comprise removing the one or more suspensions from the spheroid prior to depositing the gel. That is to say, after the spheroid is formed but before the gel is deposited, any remaining suspension may be evaporated to remove any unnecessary or excess moisture. The evaporation may be carried out in the presence of 5 vol % CO₂ at 37° C.

In various embodiments, the gel may be a hydrogel. The gel may comprise collagen, gelatin methacryloyl, and/or matrigel. Any suitable type of gel, or even any suitable extracellular matrix (ECM) material, that does not compromise the spheroid and cells may be used.

The method may further comprise subjecting the gel to a temperature of 30 to 40° C. 30 to 35° C. 35 to 40° ° C., etc., to render crosslinking within the gel for encapsulating the spheroid after arranging the frame against the substrate.

The method may further comprise coating a layer of adhesive after crosslinking of the gel. In various embodiments, the adhesive may comprise polydopamine, fibronectin, collagen, poly-L-lysine, gelatin, or any other hydrogel and extracellular matrix material suitable as an adhesive in the context of the present disclosure can be used. As a non-limiting example, polydopamine and/or fibronectin can be used as the adhesive for human umbilical vein endothelial cells (HUVEC). In certain non-limiting instances, polydopamine can be used as the adhesive for spheroids derived from HUVEC. In certain non-limiting instances, fibronection may be used as the adhesive.

The method may further comprise removing the frame from the substrate after the gel encapsulated the spheroid for retrieving the gel-encapsulated spheroid.

The present disclosure, as mentioned above, also provides for a device configured to render a spheroid encapsulated in a gel. The present device may be exchangeably termed herein a “platform”, a “microchip”, and a “microarray”. Embodiments and advantages described for the present method of the first aspect can be analogously valid for the present device subsequently described herein, and vice versa. As the various embodiments and advantages have already been described above and examples demonstrated herein, they shall not be iterated for brevity.

The device comprises a frame comprising a base and an island protruding from the base, and a substrate, wherein the frame is arrangeable against the substrate with the base distally positioned from the substrate (i) to have the gel confined between the island and the substrate and (ii) to have the gel encapsulate the spheroid.

In various embodiments, the frame may comprise two supporting structures each configured at opposing edges of the base and extending therefrom, and wherein the island is (i) configured between the two supporting structures, (ii) extends in the same direction as the two supporting structures from the base, and (iii) is vertically shorter than the two supporting structures. A non-limiting example of such a frame is illustrated in FIGS. 1A and 1B.

In certain non-limiting embodiments, the frame may further comprise two depressions each residing between one of the two supporting structures and the island. This helps to render a CBV effect when the frame is pressed against the substrate, as the narrower gap between the island and the substrate in turn renders sufficient surface tension to have a gel therein confined in place.

In various embodiments, the two supporting structures extend at least 150 μm, at least 300 μm, at least 400 μm, at least 500 μm, at least 530 μm, etc., from the base.

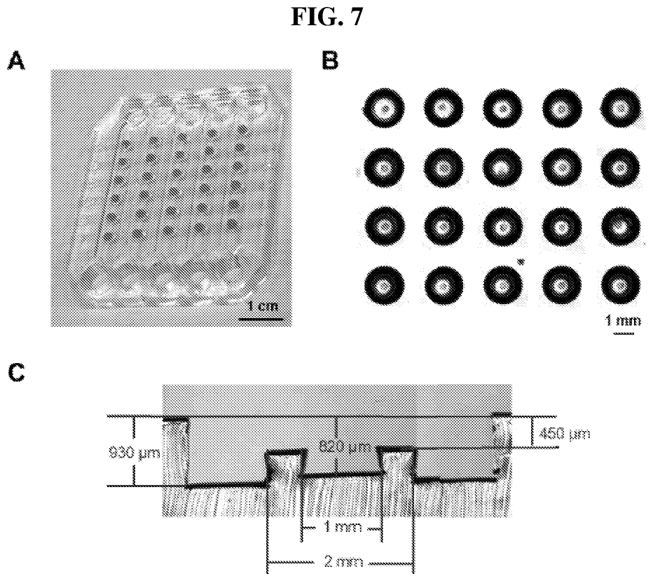

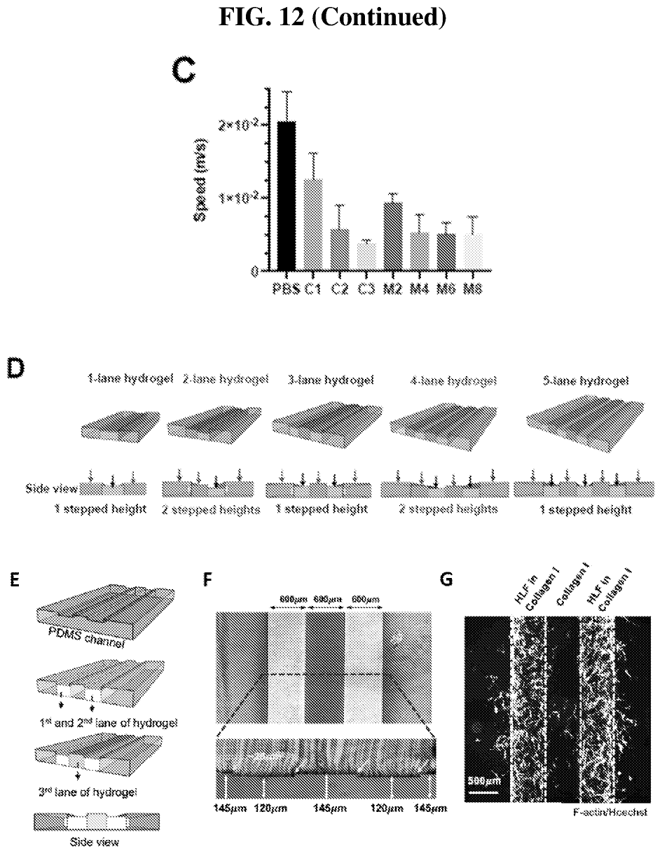

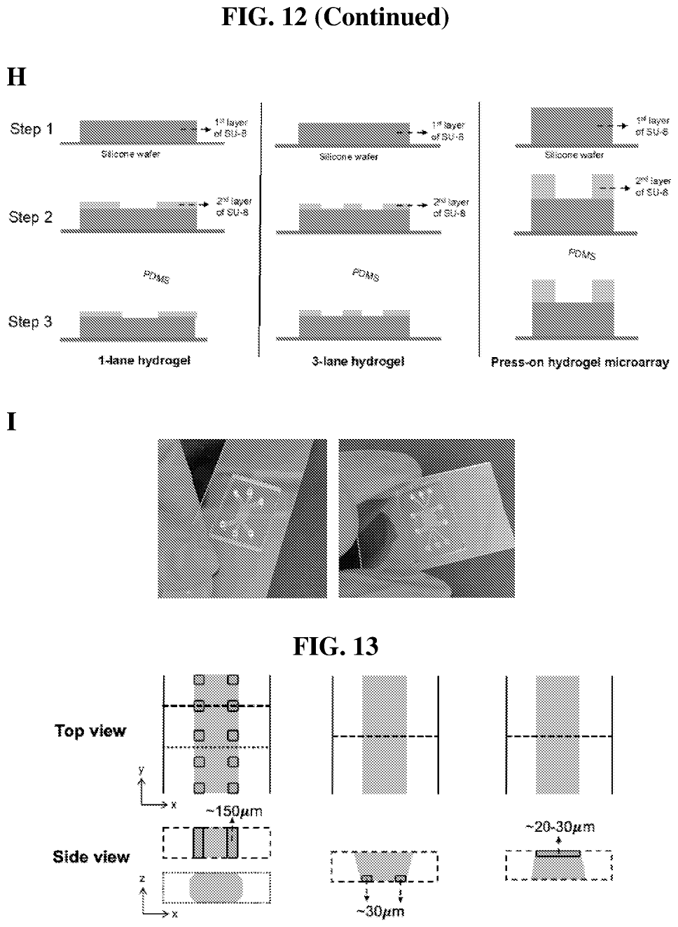

In various embodiments, the island may comprise one channel, or more than one channel, wherein the more than one channel has the same or different depth. Non-limiting examples of a frame having such an island is illustrated in FIG. 5, FIGS. 7A and 7C, 12A, rightmost image in FIG. 12D, etc. In certain non-limiting embodiments, the channel, or the more than one channel, may include one or more microwells for deposition of a cell culture (i.e. the one or more suspension of cells).

In certain non-limiting embodiments, the island may comprise at least one channel defined by multiple depressions, wherein each of the multiple depressions has a different depth. Non-limiting examples of this are illustrated in 2nd and 4th image (counting from left) of FIG. 12D. Such difference in depth for the multiple depressions in the channel may be referred to as having a “stepped height”.

In various non-limiting embodiments, the island may protrude from the base at a height at least 340 μm. In various non-limiting embodiments, the island may protrude from the base at a height in the range of 10 μm to 500 μm, 100 μm to 500 μm, 200 μm to 500 μm, 300 μm to 500 μm, 400 μm to 500 μm, 10 μm to 340 μm, 340 μm to 500 μm, etc.

In various embodiments, the island, when viewed from top down, may have or

may comprise a circular shape, a three-sided shape, a four-sided shape, or a five-sided shape. For example, the island may be circular, or semi-circular, a triangle (e.g. equilateral triangle), a rectangle, a square, a pentagon, etc.

In various embodiments, wherein the channel, or the more than one channel, in the island may be linear or curved. In various embodiments, the channel, or the more than one channel, may include one or more microwells for deposition of a cell culture (i.e. the one or more suspension of cells).

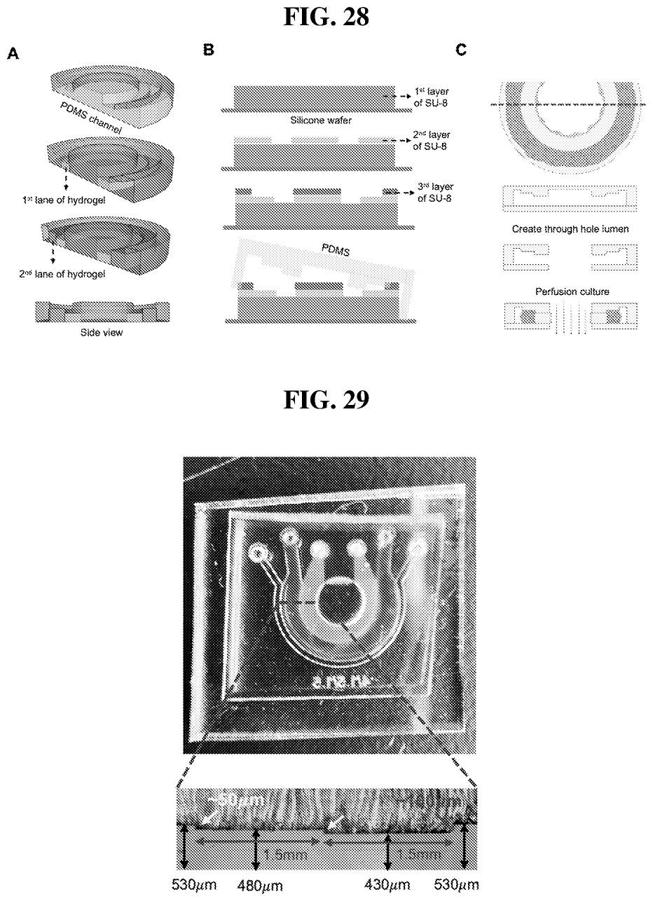

In certain non-limiting embodiments, the island may comprise at least one channel defined by multiple depressions, wherein each of the multiple depressions has a different depth, wherein the at least one channel extends horizontally across the island at a constant radius from a point in the island. In such non-limiting embodiments, the the island may comprise a lumen which extends vertically through the island. Non-limiting examples of such embodiments are illustrated via FIG. 28A to 28C.

The word “substantially” does not exclude “completely” e.g. a composition which is “substantially free” from Y may be completely free from Y. Where necessary, the word “substantially” may be omitted from the definition of the present disclosure.

In the context of various embodiments, the articles “a”, “an” and “the” as used with regard to a feature or element include a reference to one or more of the features or elements.

In the context of various embodiments, the symbol “˜”, the terms “about” and “approximately”, as applied to a numeric value encompasses the exact value and a reasonable variance.

As used herein, the term “and/or” includes any and all combinations of one or more of the associated listed items.

Unless specified otherwise, the terms “comprising” and “comprise”, and grammatical variants thereof, are intended to represent “open” or “inclusive” language such that they include recited elements but also permit inclusion of additional, unrecited elements.

### EXAMPLES

The present disclosure relates to a facile strategy for localized encapsulation of single spheroids within a defined hydrogel pattern in a microfluidic culture platform (exchangeably termed herein a “device”). The platform allows 3D culture of spheroids in a microarray using the capillary burst valve (CBV)-based hydrogel patterning technique with the versatility to establish co-culture with other cell types. The present platform can be readily scaled up for high throughput studies, and for use in potential applications in the field of organ-on-a-chip, regenerative medicine (stem-cell derived organoids), and cancer research including mechanistic studies and predictive drug screenings in a more physiologically relevant context.

Various examples of the present disclosure demonstrate a scalable microfluidic culture platform for localized formation and encapsulation of spheroids within geometrically defined hydrogel. The platform facilitates the initial confinement of a cell-laden droplet to form spheroid by hanging drop culture. Next, in-place hydrogel encapsulation of spheroids is performed based on the capillary burst valve (CBV) effect. Using the present approach, robust spheroid-ingel culture was achieved which paves the way for improved preclinical drug screening studies.

Various examples of the present disclosure may rely on a hydrogel patterning method based on channel demonstrate stepped heights and capillary burst valve (CBV) effect for blood vessel studies. The various examples further explore the versatility and scalability of the stepped-height based technique for 3D cell cultures in enclosed microchannels and spheroid-in-gel cultures in an open-channel microarray format. The various examples demonstrate hydrogel (Collagen I) patterning in a parallel lane configuration, which can be multiplexed using either one or two stepped height features.

A microarray chip for spheroid-in-gel culture using a single step “press-on” hydrogel confinement method was developed. The initial formation of breast cancer (MCF-7) spheroids was achieved using an on-chip hanging drop culture, following which each spheroid was directly encapsulated on-chip within individual hydrogel islands at the same location. Lastly, spheroids with endothelial cells (HUVEC) were co-cultured to form a vascular layer surrounding the spheroid ECM region and demonstrated cancer drug testing (paclitaxel) in this model. Taken together, the developed hydrogel patterning method is easy to fabricate by standard photolithography and soft lithography, simple to use, and can be readily adapted for use in open channels for high-throughput 3D spheroid assays.

The present method and device are described in further details, by way of non-limiting examples, as set forth below.

### Example 1A: CBV-Based Press-on Hydrogel Confinement

Examples 1A to 1D demonstrate a non-limiting example of the present device and method for encapsulating spheroid in a gel.

Based on a CBV-based ECM patterning method, hydrogel can be confined in a designated channel bounded by a sudden expansion of channel height at z-axis. Here, the example demonstrates a method to enable single step and rapid press-on hydrogel confinement that opens up possibilities for more applications including spheroid formation and in-place hydrogel encapsulation in a microarray format. The modified configuration contains a hydrogel ‘island’ that extrudes from the ceiling of the main channel. Firstly, hydrogel is loaded and confined on the island, following which the device is flipped over and pressed onto a glass slide to seal the channel. The hydrogel remains confined within the island owing to the CBV effect which is resulted from channel height difference (FIG. 1A). To fabricate the step height required for achieving the CBV effect, a two-step photo lithography was implemented to create the mold, following which standard polydimethylsiloxane (PDMS) soft lithography was used to fabricate the chip (FIG. 1B). The two-step photolithography adopted here allows for a single step of PDMS replica molding to obtain the device. This fabrication method is less laborious and more robust compared with a previously developed method, wherein two pieces of PDMS with different channel designs were manually aligned and bonded together to create the step height. For robust gel loading and confinement, the channel height for the main channel and hydrogel island is designed to be, for example, ˜530 μm, and ˜340 μm, respectively, resulting in a step height of ˜190 μm (FIG. 1C). Successful gel loading and press-on gel confinement was demonstrated using Collagen I (3 mg/ml) mixed with food dye (FIG. 1C). Prior to gel loading, the PDMS surface was made hydrophilic by 1-minute plasma treatment using a plasma cleaner (Harrick) to ensure even spreading of hydrogel onto the surface of the island.

### Example 1B: Geometrical Requirements for Hydrogel Patterning

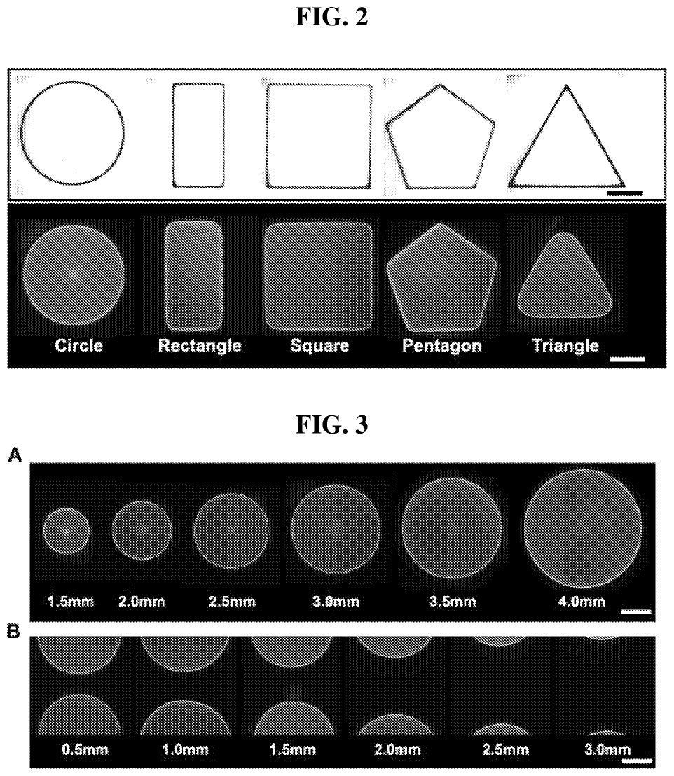

To explore the versatility of different device configuration for hydrogel patterning, first varied the shape of the hydrogel island. Results showed that hydrogel patterning was successful for different shapes including circle, square, rectangle and pentagon but was less desirable (albeit still workable) for shapes with angle of less than 60 degree (e.g. equilateral triangle) (FIG. 2). For the equilateral triangle, a patterned hydrogel with a fillet radius of 0.6 mm was formed.

The dimensional parameters including diameter and edge distance for the circular hydrogel island were further varied. It was shown that hydrogel can be successfully patterned for circular diameter above 1.5 mm (FIG. 3A), and adjacent patterned hydrogel did not cross-over with edge distance greater than 0.5 mm (FIG. 3B).

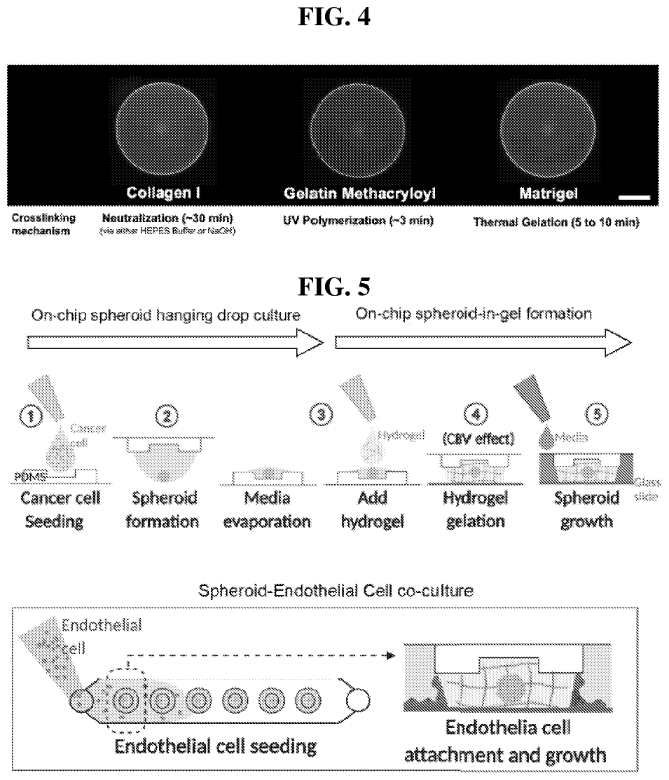

Additionally, hydrogels with different crosslinking mechanism were demonstrated to be successfully patterned on the circular island (FIG. 4).

### Example 1C: Workflow of On-Chip Spheroid-In-Gel Formation

One of the common approaches to make 3D cancer spheroid is the hanging drop method. Briefly, a droplet of cancer cell suspension is spotted on petri dish cover and inverted to allow for cell aggregation and spheroid formation by gravity. Here, the present examples demonstrated that the cancer cell suspension can be directly loaded onto the micropatterned islands of the PDMS device and form spheroid after inverting the device for hanging drop culture. Following that, hydrogel is loaded to encapsulate the spheroid after media evaporation. The chip is then inverted and sealed onto a glass slide whereby the hydrogel containing the spheroid remain confined due to the CBV effect. Lastly, culture media is added into the main channel upon gelation of the spheroid-laden hydrogel, forming a spheroid-in-gel microarray. Alternatively, co-culture can be performed on the chip by introducing another cell type (either adherent or suspension cells) into the channel. To allow for clear visualization of the media evaporation process, the island can be further designed to include a centralized microwell (FIG. 5).

### Example 1D: Investigation and Discussion of Results for On-Chip Spheroid Hanging Drop Culture

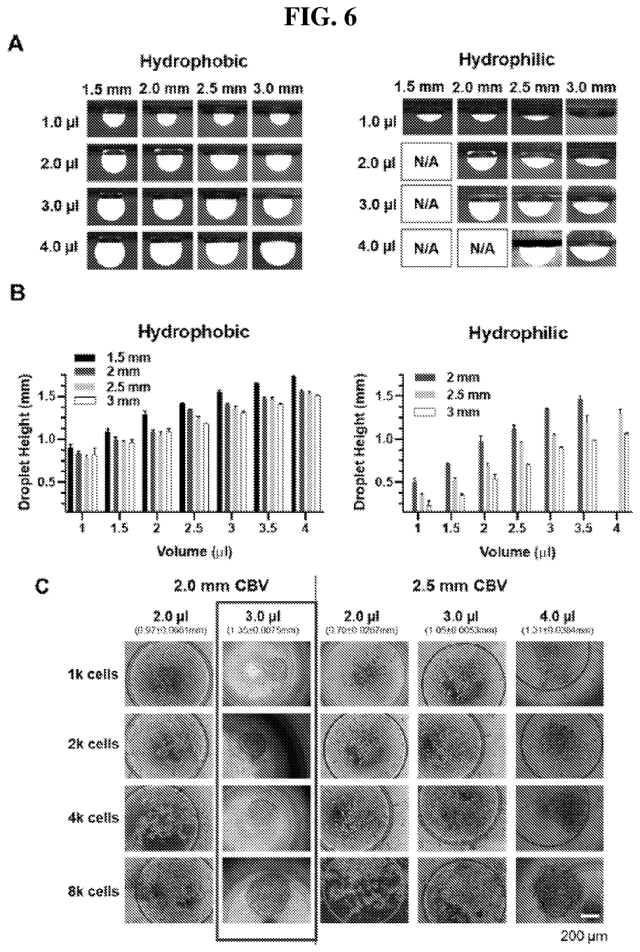

Investigation on whether the height of the droplet is configurable by varying the volume of the droplet and the diameter of the circular island was carried out. Droplet formation was characterized on both hydrophobic and hydrophilic PDMS surfaces. While hydrophobic surface allows for droplet formation on island of diameter greater than 1.5 mm for the four volumes investigated (1 ul, 2 ul, 3 ul and 4 ul), hydrophilic surface only allows for droplet formation on island with a diameter of above 2.0 mm (FIG. 6A). More importantly, a wider range of droplet height can be achieved with hydrophilic surfaces due to the increased tendency of liquid to spread onto the entire island surface (FIG. 6B). Therefore, hydrophilic surface is used for further experiments owing to the wider range of achievable droplet heights. Next, investigation on the required cell number and droplet height for successful on-chip formation of spheroid using a breast cancer cell line (MCF-7) was carried out. The chip was plasma treated and sterilized under UV for 30 min before use. A defined amount of MCF-7 cell suspension was first loaded onto the island and inverted on top of a water reservoir for hanging drop culture, following which spheroid formation was examined after two days. Interestingly, it was observed that formation of spheroid required a minimal droplet height of 1.35±0.0075 mm regardless of cell number (FIG. 6C). Therefore, 3 ul of cell suspension on circular platform of 2 mm diameter for spheroid formation was adopted. To facilitate high throughput studies, the finalized configuration of spheroid-in-gel chip consists of 5 parallel main channels, whereby each channel contains 6 islands (FIG. 7A). Colored water was used to demonstrate the successful confinement of water droplets within the islands. Spheroid formation on chip was also shown to be successful (FIG. 7B). Each island is designed to include a centralized microwell of 1 mm diameter to allow for clear visualization during media evaporation. The channel height for main channel is fabricated to be ˜930 μm, while the channel height for microwell is ˜820 μm, which allows for accommodation of various sizes of spheroids (FIG. 7C).

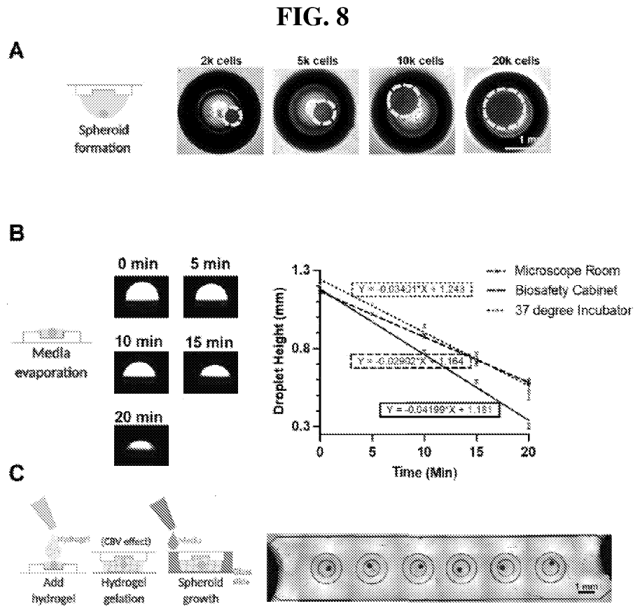

It was further demonstrated that spheroid of varying cell number can be formed on chip (FIG. 8A). Next, the media evaporation rate in three different environments including the 37-degree 5%-CO₂ incubator, biosafety cabinet and microscope room was studied. After 20 min, all three environments showed a decline of droplet height to at least 50% with biosafety cabinet having the highest evaporation rate (FIG. 8B). After the droplet evaporated to the brim of the microwell, 2 ul of collagen I (3 mg/ml) was added onto the island to encapsulate the spheroid following which the chip was inverted and sealed onto a sterilized glass slide. The chip was then transferred to the 37° C. incubator to allow the collagen to crosslink, following which media was injected into the chip to supplement the spheroid (FIG. 8C).

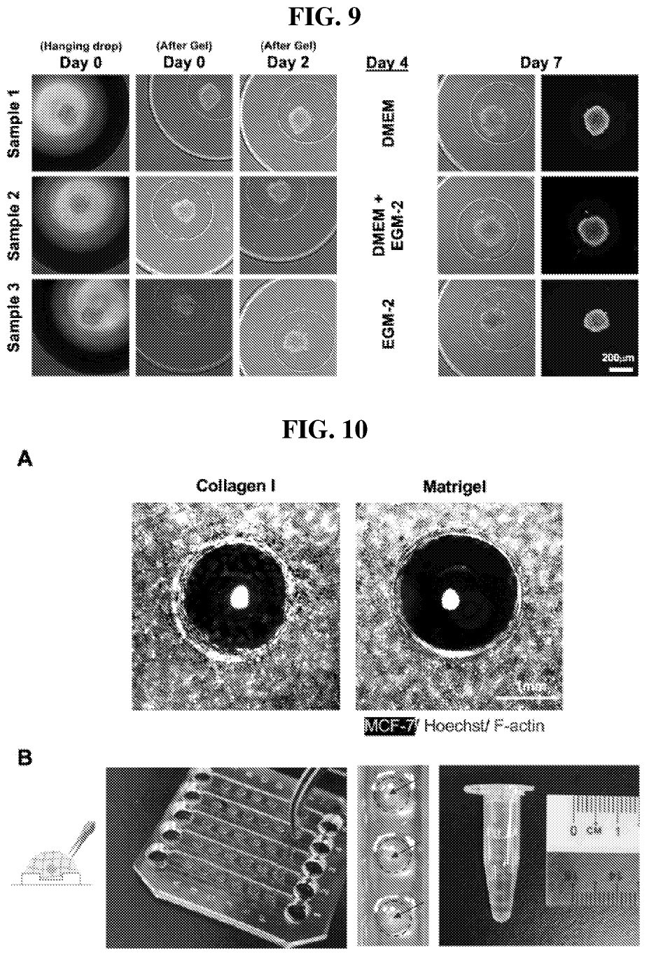

To investigate the feasibility to perform co-culture on the chip, different culture medium was introduced into the channel, following which live/dead assay was performed to examine the viability of the spheroid. As a result, the spheroid showed minimal cell death over a period of 7 days for all the three media tested (FIG. 9).

Co-culture of human umbilical vein endothelial cells (HUVEC) and MCF-7 spheroid was performed on the chip to recapitulate the vascular network surrounding the tumor as observed in vivo. Upon crosslinking of hydrogel, the channel was coated with polydopamine (1 mg/ml) to enhance cell adhesion, following which HUVEC suspension (1.5 million cells per ml) was loaded into the channel. A confluent layer of HUVEC was formed after 2 days for both collagen I and Matrigel (FIG. 10A). Additionally, the chip can be easily peeled off from the glass slide for retrieving the spheroid-laden gel if needed (FIG. 10B).

### Example 2A: Another Non-Limiting Example of Device Fabrication and Cell Culture

The frame (which is a component of the device, the other component being the substrate) was fabricated using standard photolithography and soft lithography. In brief, polydimethylsiloxane (PDMS) prepolymer was mixed with curing agent (Dow Corning, Midland, MI, USA) at the ratio of 10:1 (w/w) and poured onto the patterned silicon wafer mold, degassed for 30 min and cured for 2 hr at 75° C. The PDMS slab was cut out and retrieved from the mold, following which a biopsy puncher was used to create the inlet and outlet for the device. For the lane configuration chips, the PDMS slab was plasma bonded to a glass slide using a plasma cleaner (PDC-002, Harrick Plasma Inc, Ithaca, NY, USA). The bonded device was kept at 75° C. overnight to enhance bonding and allow for hydrophobic recovery. Devices were sterilized by ultraviolet light for 30 min prior to on-chip cell culture experiments.

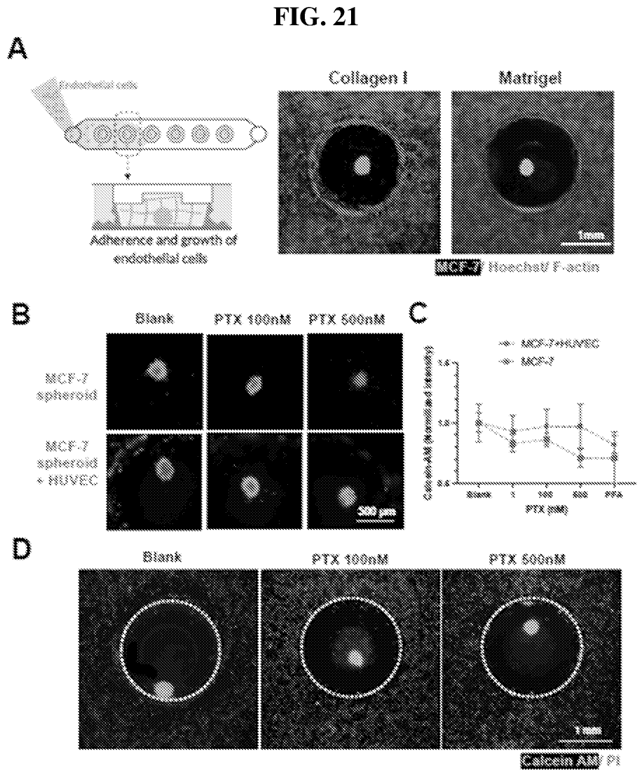

For cell culture, HUVECs were maintained using Endothelial Cell Growth Medium-2 (EGM-2) BulletKit (Lonza, Basel, Switzerland) supplemented with 1% Penicillin-Streptomycin (P/S). Human Lung Fibroblasts (HLF) were maintained using FGMTM-2 Fibroblast Growth Medium-2 (FGM-2) BulletKit (Lonza). Human breast cancer cells (MCF-7) were maintained using Dulbecco's Modified Eagle Medium (DMEM) (Gibco, Life Technologies, Carlsbad, CA, USA) supplemented with 10% Fetal Bovine Serum (FBS) (Gibco) and 1% P/S. The cells were maintained at 37° C. in a humidified 5% CO₂ incubator and passaged using 0.25% trypsin with 1 mM EDTA (Gibco) upon confluency. Passage numbers 3 to 10 were used for HUVEC (FIG. 21A to 21D) and HLF (FIG. 12G).

### Example 2B: On-Chip Cell Culture in Lane Format Chips for Example 2A

For cell seeding into the lane configuration chips, collagen Type I (3 mg/mL) (rat tail, Corning, NY, USA) was prepared, following which the gel was loaded into the chip and allowed to crosslink at 37° C. for 30 min. For HUVEC seeding into the one-lane hydrogel chip, the fluidic channels were coated with 50 g/mL of fibronectin (Sigma Aldrich, St. Louis, MO, USA) for 30 min at 37° C. prior to cell seeding. HUVECs were dissociated and resuspended to a concentration of 2 million cells per mL, following which the suspension was loaded into the fluidic channel and the HUVECs were allowed to grow until confluency.

For HLF seeding into the three-lane hydrogel chip, HLF were dissociated and resuspended into collagen I (3 mg/mL) to a concentration of 1 million cells per mL. The HLF-laden collagen was loaded into the two side hydrogel channels and allowed to crosslink at 37° ° C. for 30 min, following which the center hydrogel channel was filled with cell-free collagen. Lastly, the two fluidic channels were loaded with FGM-2 to replenish the cells. The chip was incubated at 37° C. for 2 days before fixation and imaging.

### Example 2C: On-Chip Spheroid-In-Gel Culture in Microarray Chip for Example 2A

For spheroid-in-gel culture on the microarray chip, the PDMS surface was made hydrophilic by a 1-min plasma treatment using a plasma cleaner (PDC-002, Harrick Plasma Inc, Ithaca, NY, USA) to ensure even spreading of hydrogel onto the surface of the island. MCF-7 cells were dissociated and resuspended to a concentration of 1.7 million per mL. Collagen I was added to the suspension at 20 g/mL to enhance spheroid formation. MCF-7 suspension was loaded onto each island (3 L per island), following which the PDMS chip was inverted on top of a water reservoir for a hanging drop culture.

For investigating the number of cells and droplet height for successful on-chip formation of spheroid, the concentration and volume of the MCF-7 suspension were varied accordingly. The chip was maintained 37ºC for 2 days to allow for spheroid formation. In certain selected experiments, the MCF-7 cells were labelled with the Vybrant™ DiO Cell-Labeling Solution (Thermo Fisher, Waltham, MA, USA) prior to seeding onto the chip. Upon spheroid formation, the chip was subjected to media evaporation in the 37° C. incubator, following which collagen I (3 mg/mL) or Matrigel (4 mg/mL, Corning. NY, USA) was loaded onto each island (2 L per island) for in-place encapsulation of the spheroid.

The chip was incubated at 37° C. for 30 min to allow for gelation of collagen and Matrigel, following which DMEM with 10% FBS was added into the fluidic channel. For co-culture with HUVEC, the channel was coated with polydopamine (1 mg/mL) for 30 min at 37° ° C. to facilitate HUVEC adhesion and growth. The polydopamine solution was prepared by dissolving dopamine hydrochloride (Sigma Aldrich, St. Louis, MO, USA) in Tris-HCl buffer, pH 8.5. The channel was washed with 1× Phosphate Buffer Saline (PBS) three times, following which a suspension of HUVEC (1.5 million/mL) was loaded into the channel and allowed to grow for 2 days to reach confluency. The HUVEC were cultivated under static conditions.

### Example 2D: Immunostaining, Drug Treatment and Live/Dead Assay for Example 2A

The chip was washed with 1×PBS and fixed with 4% paraformaldehyde (PFA) (Sigma Aldrich, St. Louis, MO, USA) for 15 min, following which the cells were stained with AlexaFlour 568 phalloidin (0.17 μm, Life Technologies, Carlsbad, CA, USA) and Hoechst 33342 (1 μg/mL, Life Technologies, Carlsbad, CA, USA) by incubating at room temperature for 45 min. For staining of HUVEC with VE-Cadherin, the cells were permeabilized with 0.1% Triton-X 100 in PBS for 15 min, washed three times with 1×PBS, and blocked with 0.5% bovine serum albumin (BSA) in PBS for 2 hr at room temperature.

Following that, the cells were stained with VE-Cadherin rabbit anti-human CD144 primary antibody (10 μg/mL, Enzo, Farmingdale, NY, USA) overnight at 4° C. On the next day, the cells were rinsed with 0.1% BSA in PBS three times, and fluorescently labeled with AlexaFluor 488 goat anti-rabbit secondary antibody (20 μg/mL, Life Technologies, Carlsbad, CA, USA) for 4 hr at room temperature. For live/dead assay of spheroids, the chip was washed with 1×PBS and stained with Calcein-AM (0.4 μM Life Technologies, Carlsbad, CA, USA), Propidium Iodide (PI) (2 μg/mL, Biolegend), and Hoechst-33342 (1 μg/mL, Life Technologies, Carlsbad, CA. USA) at 37° C. for 30 min.

The cells/spheroids were then imaged using a fluorescence microscope (Nikon Eclipse Ti, Melville, NY, USA). For drug treatment, once HUVEC reach confluency, Paclitaxel (Thermo Fisher, Waltham, MA, USA) was added into the fluidic channels at concentrations of 100 and 500 nM, following which the chip was cultured for 3 days before a live/dead assay. Spheroid fixed with 4% PFA was used as negative controls. Fluorescence intensity of Calcein-AM and PI was analyzed using ImageJ and calculated according to equation (1) below.

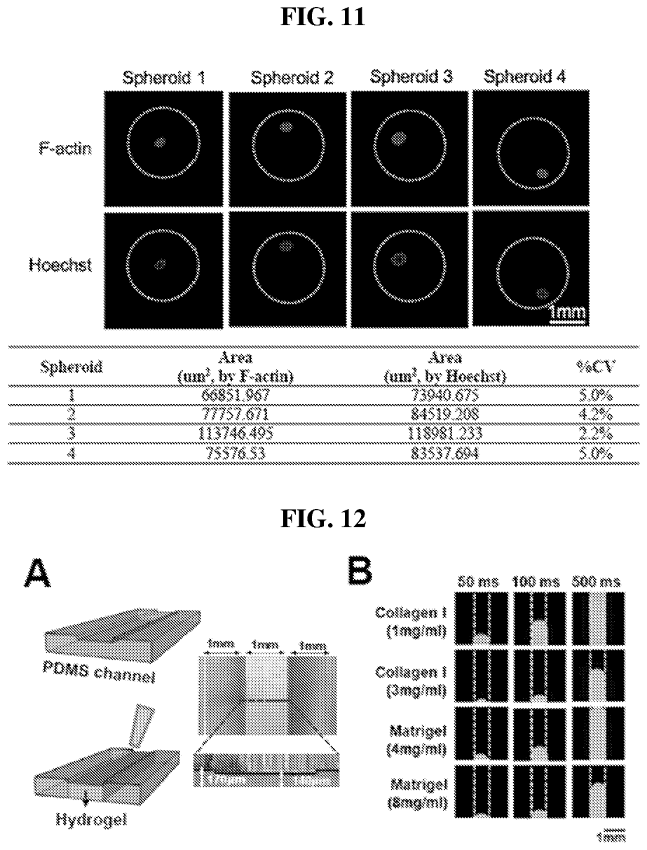

I=(Iₜ−I_{b})/A (1)
where Iₜ is the integrated intensity, I_{b} is the background intensity, and A is the spheroid area. Spheroid area is determined by Hoechst staining. Quantification of spheroid area by Hoechst staining and F-actin staining was found to be similar (FIG. 11). It was estimated by measuring the mean gray value of regions without cells and multiplying this value to the area of image. The intensity of Calcein-AM was normalized to the untreated group of each experiment. The same set of imaging parameters including exposure time and focal plane was used when acquiring the images to minimize inaccuracies in the fluorescence intensity.

### Example 2E: Endothelial Barrier Integrity Study for Example 2A

To assess the endothelial barrier integrity after drug treatment in the spheroid-in-gel chip, Collagen I (3 mg/mL) and HUVEC were seeded into the chip as described in example 2C. Sub-confluent HUVEC were treated with Paclitaxel (100 and 500 nM) and cultured for 3 days prior to the barrier permeability test. Fluorescence images were taken before and after 1 hr incubation with 10 μg/mL 70 kDa dextran conjugated with fluorescein isothiocyanate (FITC, Sigma Aldrich, St. Louis, MO, USA) loaded in the fluidic channels. Fluorescence intensities within the hydrogel island were expressed as the fold change (time 60 min over 0 min) and normalized to the untreated control.

### Example 3A: Discussion on Results for Stepped Height-Based Hydrogel Patterning in Enclosed Microchannels for Example 2A

ECM/hydrogel can be confined in designated microchannel regions by the sudden expansion of channel height without any micropillars or microstructures. Briefly, with a two-layered PDMS device and a stepped height feature at the intersection of a hydrogel channel and fluidic channel, the ECM introduced at the bottom layer is confined at the stepped height owing to the CBV effect conferred by the sudden channel expansion along the Z-axis.

Here, the scalability of the present technique to pattern multiple (>2) adjacent lanes of hydrogel was explored. Using the two-step photolithography method, a microfluidic with 3 parallel channels and a stepped height feature of ˜30 μm (yellow arrows) was fabricated for hydrogel confinement (Collagen I) within the shallower channel (middle) (FIG. 12A, 12H, 12I). Endothelial cells (HUVEC) were subsequently cultured in the two fluidic channels on both sides of the collagen to construct 3D endothelial barriers as a proof-of-concept for organ-on-chip applications (FIG. 12G).

Next, the patterning of different hydrogels was investigated by characterizing the loading speed for 1×PBS, Collagen I (1, 2, and 3 mg/mL), and Matrigel (2, 4, 6, and 8 mg/mL). All the tested gel conditions were successfully confined in the present device, thus, demonstrating the versatility of the present technique. As expected, both collagen and Matrigel exhibited a concentration-dependent decrease in loading speed due to higher viscosities, which ranged from ˜3.78×10⁻³ to 2.04×10⁻² m/s (FIGS. 12B and 12C).

Micropillar-based multiple-lane hydrogel patterning has been reported by others for co-culture or modeling microenvironment (see FIG. 13 leftmost image). Herein, the present examples demonstrated the use of stepped height for patterning multiple lanes of hydrogel. Based on the hydrogel patterning principle, a single stepped height feature was considered for odd number of hydrogel lanes, while two stepped height features were considered for even number of hydrogel lanes in a microfluidic design with fluidic channels at both sides (FIG. 12D). To exemplify this, a 3-lane hydrogel chip with a single stepped height (˜25 μm) was fabricated (FIG. 12H, 12I). Sequential patterning of 3 hydrogel lanes (collagen filled with red or green dye) was achieved (FIG. 12E, 12F), and the chip was used to demonstrate 3D cell culture by patterning two human lung fibroblasts (HLF)-containing hydrogel lanes with a cell-free hydrogel sandwiched in between them (FIG. 12G), a platform which is potentially useful for 3D cell migration assays.

### Example 3B: Discussion on Results for Rapid “Press-On” Hydrogel Patterning in Open-Channel Microarray Chip for Example 2A

The present method was adapted for single step “press-on” hydrogel confinement in an open-channel using a micropatterned PDMS substrate and glass slide. The PDMS substrate is first patterned with extruding stepped height features required for gel patterning to form individual “hydrogel islands”. Briefly, hydrogel droplets are deposited on the extruded features (island) of the open PDMS surface, following which the device is flipped over and pressed onto a glass slide.

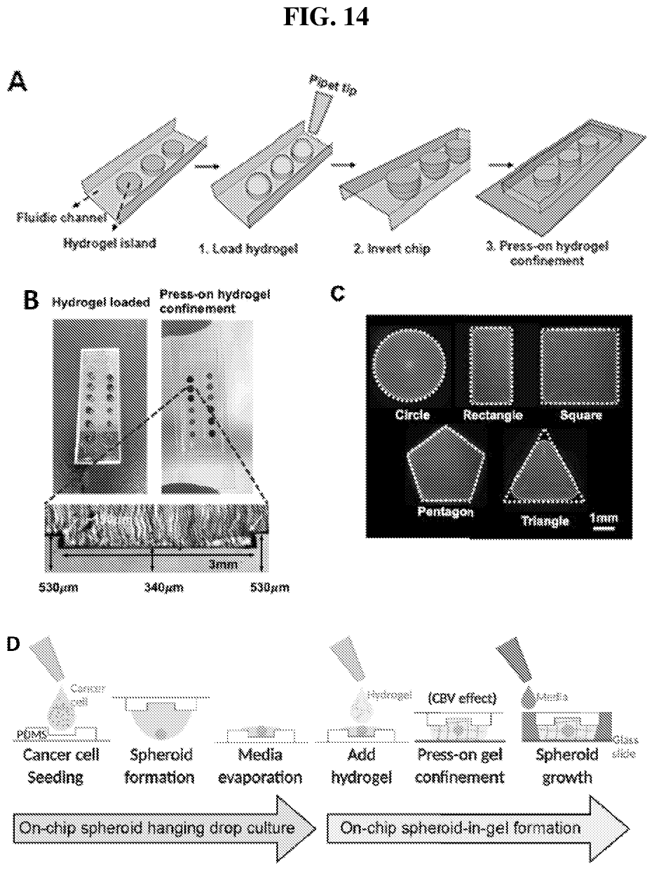

Upon contact, the hydrogel remains confined within the island due to the surface tension resulted from the channel height differences (FIG. 14A). For robust gel loading and press-on gel confinement, the channel heights for the main channel and hydrogel island were designed to be ˜530 and ˜340 μm, respectively, resulting in a step height of ˜190 μm (FIG. 14B). The deposited gel volume is calculated based on arci of each island and channel height (˜2.5 μL).

Successful gel loading and press-on gel confinement was demonstrated using collagen I (3 mg/mL) mixed with food dye, and the user can spot different gels on each island as desired (FIG. 14B). It should be noted that the gel loading process on open surfaces/channels is significantly easier compared with conventional gel patterning in enclosed microchannels as the user does not need to control the gel loading pressure, and the gel loading speed can be further increased with the use of electronic pipettes.

To explore the versatility for hydrogel patterning, the shape of the hydrogel islands was varied. Results showed that hydrogel patterning was successful for different shapes, including circles, squares, rectangles, and pentagons, but less considerable for shapes with angle of less than 60 degrees albeit still usable (e.g. equilateral triangles) (FIG. 14C). For the equilateral triangle, a patterned hydrogel with a fillet radius of 0.6 mm was formed, which is likely due to the surface tension properties of the hydrogel itself.

The dimensional parameters for circular islands, including the diameter and edge distance between each island was investigated. The circular island was selected for subsequent studies as it is isometric in nature and enables the formation of a concave surface (dome-shaped) that is similar to conventional hanging drop spheroid cultures. It was shown that hydrogel can be successfully patterned for circular diameters above 1.5 mm (FIG. 3A), and adjacent patterned hydrogel did not spill over with an edge distance greater than 0.5 mm (FIG. 3B).

Additionally, it was demonstrated that hydrogels (Collagen I, Matrigel, and Gelatin Methacryloyl) with different crosslinking mechanism (heat or UV) can be successfully patterned within the circular island (FIG. 4).

### Example 3C: Discussion on Results for Spheroid-In-Gel Culture Array Using Press-on Hydrogel Patterning for Example 2A

An approach to culture 3D cancer spheroid is the hanging drop method. Briefly, a droplet of cancer cell suspension is spotted on a petri dish cover and inverted to allow for cell aggregation and spheroid formation by gravity in the concave surface. Here, an on-chip hanging drop culture array was developed using the extruded island features on a PDMS substrate.

Although spheroid hanging drop culture can be performed on flat and hydrophobic PDMS surfaces, and the extruded islands are desirable to confer the stepped height to achieve hydrogel confinement due to the CBV effect upon contact with the glass substrate. Secondly, the spheroid-in-gel island array can facilitate co-culture of adherent cells (e.g., endothelial cells) outside the patterned islands in an enclosed channel.

Using the present platform, a cancer cell suspension was directly loaded onto the extruded islands and remained confined as a droplet (FIG. 14D). Upon spheroid formation in the hanging-drop culture, a portion of the culture media was removed by evaporation so that hydrogel can be deposited on the islands to encapsulate the spheroids. Once the hydrogel has crosslinked, culture media can be loaded into the channel and diffused through the hydrogel for spheroid culture. In addition to the extruded island feature, a centralized microwell was added to facilitate centralization of the spheroid in the developed spheroid-in-gel chip.

### Example 3D: Discussion on Results for On-Chip Spheroid Hanging Drop Culture for Example 2A

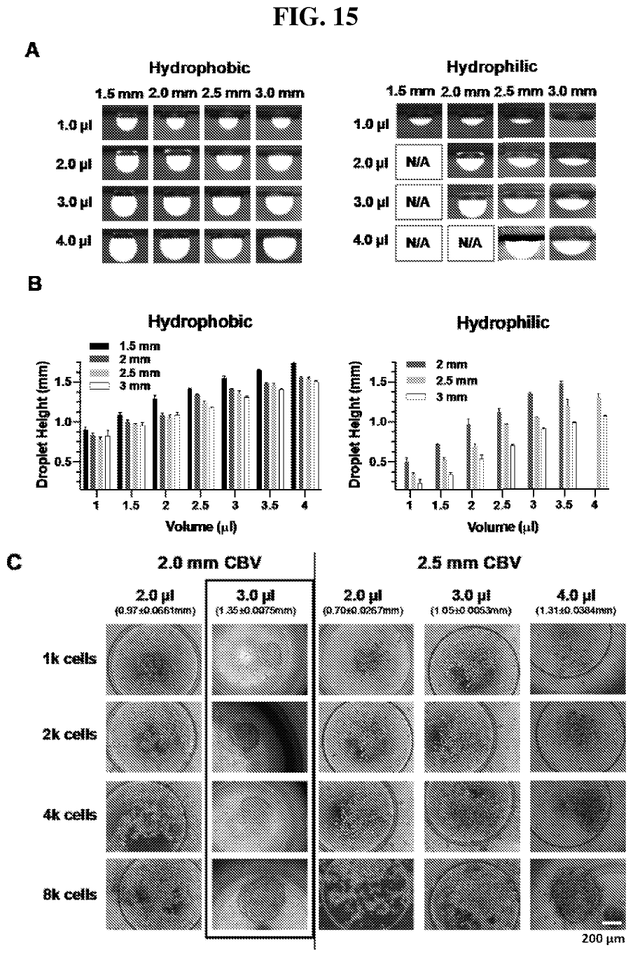

As the droplet shape may affect spheroid formation, it was first investigated if the droplet height and contact angle can be tuned by varying the droplet volume, diameter of the circular island, and the surface hydrophobicity of PDMS. The patterned islands have an additional microwell feature to facilitate spheroid centralization during manual handling of the devices. While the hydrophobic surface allowed for droplet formation on islands of diameter greater than 1.5 mm for the four volumes investigated (1, 2, 3, and 4 μL), droplets were only formed on islands with a diameter above 2 mm for hydrophilic surface (FIG. 15A).

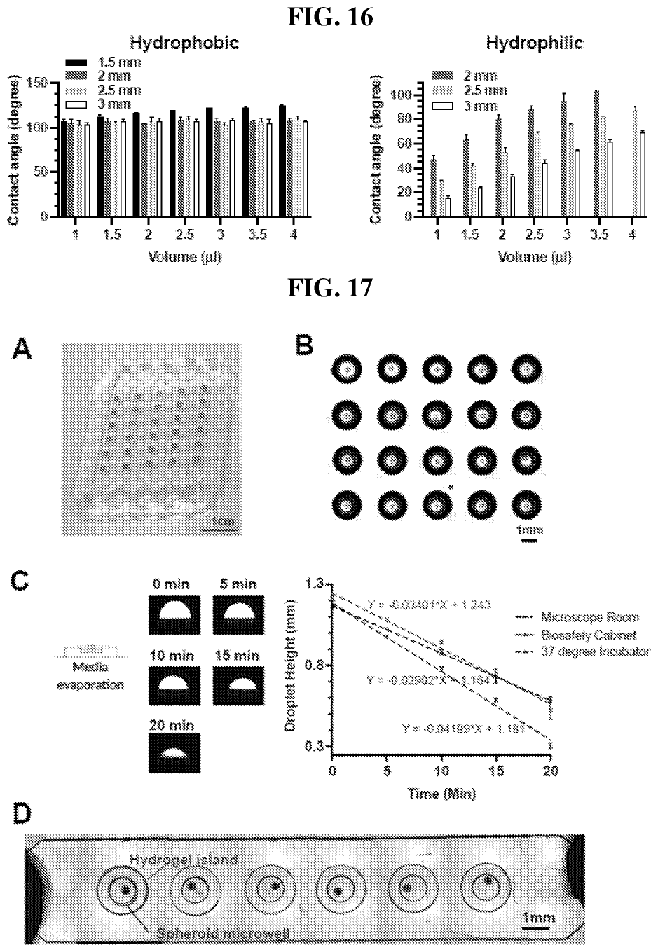

Hydrophilic surfaces also had lower droplet heights due to higher wettability (FIGS. 15B and 16). Therefore, a hydrophilic surface was used for subsequent spheroid experiments due to the wider range of achievable droplet heights and contact angle (FIG. 15B). Next, the example investigated the number of cancer cells and droplet height for successful on-chip formation of spheroid using a breast cancer cell line (MCF-7). A defined amount of MCF-7 cell suspension was first loaded onto the island and inverted for a hanging drop culture for two days. Interestingly, it was observed that the formation of a spheroid required a minimal droplet height of 1.35±0.0075 mm regardless of cell number (FIG. 15C). Therefore, the volume was set as 3 μL of cell suspension on a circular island of a 2 mm diameter for optimal spheroid formation.

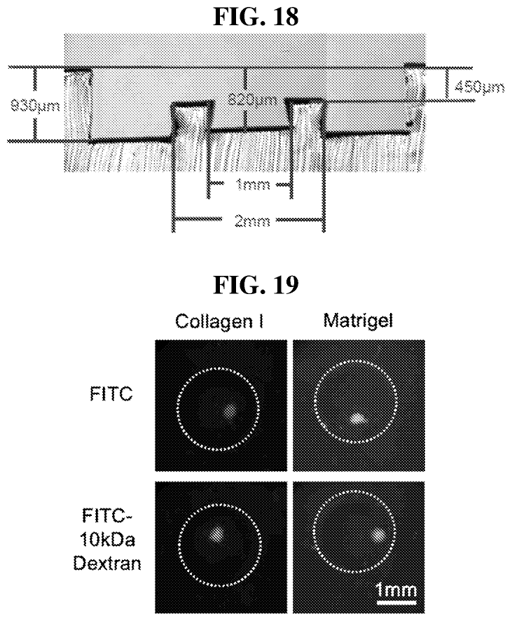

As a proof-of-concept for high throughput studies, the finalized configuration of spheroid-in-gel chip has five parallel main channels with each channel having six individual circular hydrogel islands (FIG. 17A). The channel height for the main channel was ˜930 μm, while the channel height for the microwell was ˜820 μm to accommodate various sizes of spheroids (FIG. 18). To combine the on-chip hanging drop method with stepped height-based hydrogel patterning on the same platform, the culture media on each island was removed prior to addition of hydrogel.

Upon successful spheroid formation after two days' hanging drop culture (FIG. 17B), the examples examined the media evaporation rate in three different environments, including the 37° C. 5%-CO₂ incubator, biosafety cabinet and microscope room (at room temperature). After 20 min, all three environments showed a decline of droplet height to at least 50%, with biosafety cabinet having the highest evaporation rate possibly due to additional convective air flow (FIG. 15C).

The evaporation step was important to remove a portion of the culture media before adding the hydrogel to prevent spillage out of the island features. The process was also carefully performed to ensure the spheroids remained contained within the culture media to minimize any adverse effects. It should be noted the transient increase in concentration of soluble factors in the media may affect cell metabolism, which can be further investigated.

To ensure minimal damages to the cultured spheroids, media evaporation was performed in the 37° C. incubator for the rest of the study. Next, Collagen I (2 μL) was added onto each island to encapsulate the spheroid, following which the “press-on” gel patterning was used to enclose the channel and fixate the spheroid-containing collagen against a glass slide.

Spheroids were confined within the microwell region as intended (FIG. 15D). As the spheroid position may vary within the hydrogel islands, a diffusion study was performed to investigate whether the spheroid position would affect the uptake of molecules. By incubating the spheroids with FITC (˜400 Da) and FITC-10 kDa Dextran for 24 h, it was observed that effective diffusion and uptake of both molecules into the spheroid regardless of the spheroid position (FIG. 19).

### Example 3E: Discussion on Results for Spheroid-In-Gel Formation and Co-Culture with Endothelial Cells for Example 2A

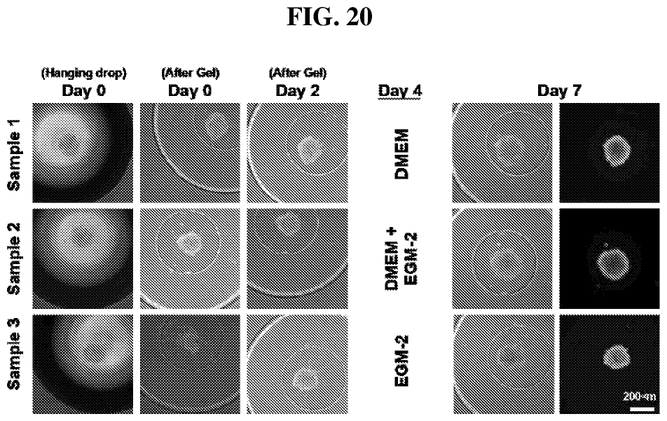

Next, the example investigated the feasibility to perform co-culture of spheroid with endothelial cells to recapitulate the vascular barrier surrounding the tumor as observed in vivo. For cell co-cultures, the different cells have to be properly replenished using a common culture media. As such, there was introduction of different culture media (DMEM (for cancer cells), EGM-2 (for endothelial cells), and DMEM+EGM-2 (1:1)) at day 4 and examined the spheroid viability at day 7. It can be observed that the spheroids showed minimal cell death over a period of 7 days for all the three media tested (FIG. 20). To ensure optimal growth of endothelial cells, the EGM-2 media was used for co-cultures of spheroid and endothelial cells.

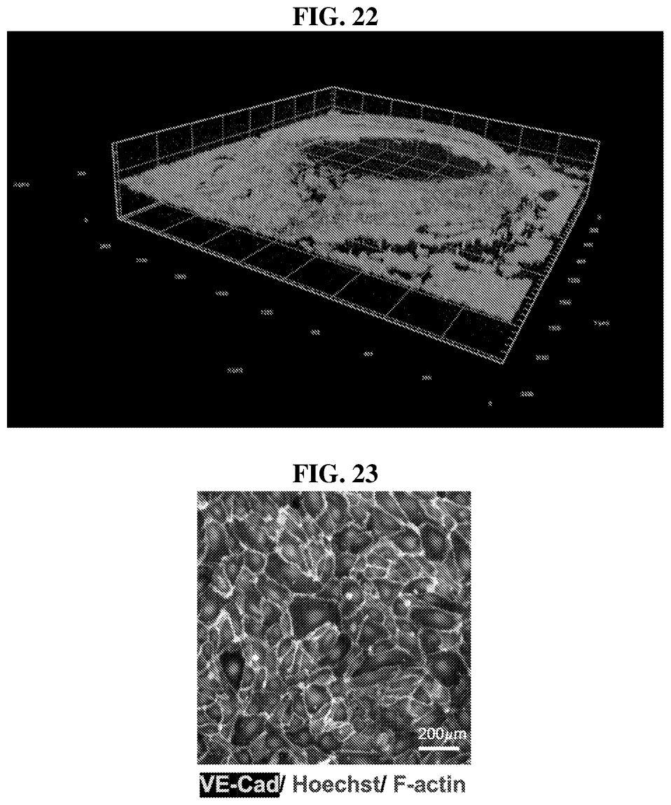

Human umbilical vein endothelial cells (HUVEC) were introduced into the fluidic channel to form a monolayer at the channel bottom and along the gel surface of the MCF-7 spheroid-in-gel islands. As expected, a confluent layer of HUVEC was formed surrounding the spheroid ECM region after 2 days (FIGS. 21A and 22), with the presence of an adherens junction marker (VE-Cadherin) (FIG. 23). This shows the feasibility to establish a spheroid-in-gel co-culture using the present platform.

As a proof-of-concept for drug screening applications, the spheroids with or without HUVEC co-culture were treated with Paclitaxel (PTX) for 3 days, following which the viability of the spheroids were examined. Live/dead staining (Calcein-AM/PI) showed higher MCF-7 death with increasing drug concentration for mono-cultured spheroids, and higher EC death for spheroid co-cultured with HUVEC (FIG. 21B, 21D). The quantification of spheroid Calcein-AM intensity further showed a trend of better survival of spheroids in co-cultures as compared to mono-culture, although no significant difference was observed.

This is possibly due to presence of endothelial barrier or paracrine effects by the surrounding HUVEC, thus suggesting the importance of performing drug screening studies in physiological microenvironment (FIG. 21C, 21D). Live/dead staining (Calcein-AM/PI) showed higher MCF-7 and EC death with increasing drug concentration (FIG. 21B, 21D). Quantification of spheroid Calcein-AM intensity showed a trend of better survival of spheroids in co-cultures as compared to mono-culture (FIG. 21C), although no significant difference was observed.

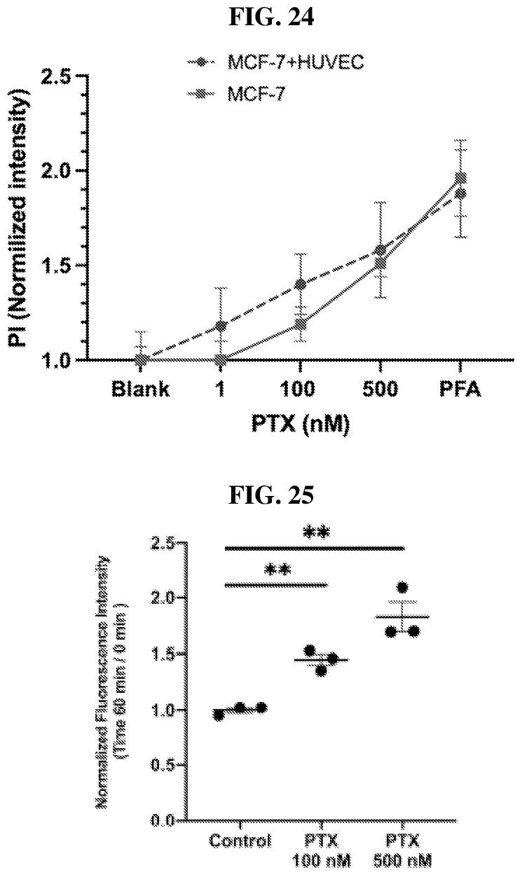

There were also negligible differences in PI intensity between drug-treated spheroids in mono-culture and co-culture (FIG. 24), which could be due to high red background noise and limited sensitivity with the 2D imaging method used. Finally, the increased EC death (FIG. 21D) also impaired the endothelial barrier integrity based on the diffusion of FITC-Dextran 70 kDa into the hydrogel islands (FIG. 25). This barrier breakdown may affect spheroid-immune cell interactions if leukocytes are present in the culture platform, thus, suggesting the important of adding an immune component to better model the tumor microenvironment as future work.

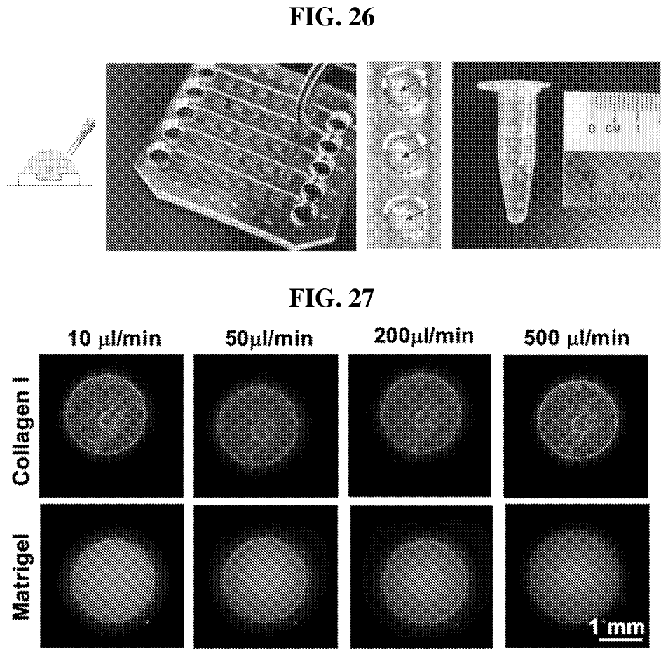

As the PDMS substrate can be separated from the glass slide due to reversible bonding in the spheroid-in-gel platform, it is demonstrated that individual spheroid-containing gels can be retrieved using a tweezer manually (FIG. 26). It is envisioned that this feature of selected spheroid and ECM retrieval is a considerable factor for downstream immuno-oncology studies to characterize both tumor and immune/perivascular cells within the ECM.

### Example 4: Summarized Discussion of Results for Example 2A

In aforesaid examples, a stepped height-based hydrogel patterning technique was developed to create different chip configurations for 3D cell cultures in enclosed microchannels and spheroid-in-gel cultures in open channels. The stepped height-based method described here uses standard photolithography to fabricate a wafer mold with multi-height patterned features and a single-step PDMS soft lithography to obtain the final device. Therefore, it can be easily adopted by research laboratories where standard photo lithography and soft lithography are widely used and easily accessible.

While the dual-lane stepped-height based hydrogel chip have been reported for arterial wall-on-a-chip, the examples further demonstrated the scalability of this method to pattern multiple adjacent lanes of hydrogel using single or dual stepped heights. An injection-molded culture platform for patterning three adjacent lanes of hydrogel have been reported, but it lacks the flexibility to pattern two different hydrogels for the first two lanes.

Additionally, the height of the side hydrogel lanes and the center one differs by 15-fold, which can potentially hamper cell-cell or cell-ECM interaction between different hydrogel lanes. Here, as the stepped height features are located at the upper PDMS channel, this minimizes technical issues related to the imaging of a cell monolayer at the channel bottom and further facilitates the integration of electrode sensors at the bottom substrate for real-time biosensing capabilities in organ-on-chip devices.

Secondly, the examples demonstrated that this stepped height-based hydrogel patterning method can be scaled-up in dimensions and adapted for operation in open channels to establish 3D spheroid-in-gel culture arrays. The culture platform demonstrated in this study was configured to have five main channels with each channel containing six islands for spheroid cultures. The configuration is easily amenable and can be scaled up as needed. Although a PDMS-based hanging drop culture platform without hydrogel patterning features has been reported, such culture system cannot be enclosed after spheroid encapsulation, and hence it is not possible to perform perfusion cultures or co-cultures of spheroids with other cell types.

Herein, using a simple and rapid press-on hydrogel confinement method, the following features including (1) versatility to pattern different hydrogel island shapes, (2) integrating on-chip spheroid formation and in-place gel encapsulation, which circumvents the need to manually transfer each spheroid into the gel, (3) flexibility to perform co-culture with other cell types, and (4) the retrieval of individual spheroid for off-chip downstream analysis, were achieved.

As shear forces play a role in endothelial functionality, it was demonstrated that the hydrogel islands will not dislodge under flow (at a flow rate up to 500 L/min for 5 min) in the presently developed platform (FIG. 27), thus, suggesting the potential to perform perfusion culture. Another potential is to distinguish the structure of the 3D spheroids in hydrogel islands using confocal imaging. Lastly, while it was demonstrated a toxicity effect after 3 days of drug treatment in this example, it is of considerable interest to perform long-term monitoring of spheroid drug responses in the future.

Taken together, the spheroid-in-gel culture platform offers compelling advantages over existing spheroid culture platforms owing to its simple fabrication and operation steps as well as the ability to preserve the biological complexity needed for physiological relevancy while remaining scalable for high throughput studies. It can be envisioned that the developed hydrogel patterning technique and platforms are of great interest for both fundamental studies and translational research, such as high content drug screenings.

### Example 5: Another Non-Limiting Example of Micropatterned 3D Hydrogel Microarray in Fluidic Channels for Spheroid-In-Gel Culture

To further explore the flexibility of the technique, hydrogel patterning in curved channels was investigated. For proof of concept, the dual-lane hydrogel chip was adapted into a curved design with 3 concentric channels surrounding a circular chamber (FIG. 28A). This configuration can be fabricated using the same 3-step photolithography method as the dual-lane hydrogel chip with straight channels (FIG. 28B). Considerably, this configuration could be useful for organ-on-a-chip applications like reconstituting the cross-section of the artery with the flexibility to perform perfusion culture (FIG. 28C). Moreover, previous report has shown surface curvature plays a role in guiding spatiotemporal cell (e.g. epithelial cell, fibroblasts, airway SMC) and tissue organization. Therefore, this configuration could also be used to study changes in cellular behaviour (growth, orientation, migration, etc) arisen from the curved interface. Further to the intended application to construct vascular model, this configuration could be useful for studying other biological phenomenon like wound healing where the interface curvature are known to play a role in providing mechanobiological cues to guide cellular behaviour.

To facilitate study on the role of shear stress, it is desirable to have more cells growing on the ECM sidewall (FIG. 28C). Hence the channel height and stepped height was fabricated to be ˜500 μm and ˜50 μm respectively for this chip. As expected, sequential hydrogel patterning was achieved in curved channels with the two hydrogel channels loaded with collagen (opaque-looking for the 1^{st} hydrogel channel, and transparent-looking for the 2nd hydrogel channel) (FIG. 29).

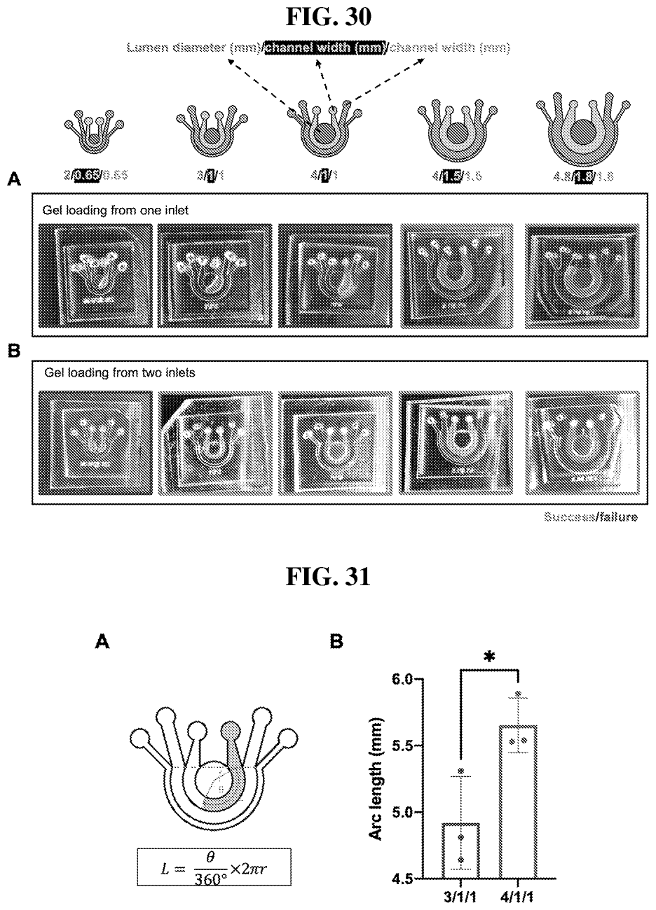

In curved channel, the traveling trajectory of gel becomes an arc. Hence the required channel width for successful hydrogel patterning may be different from that in straight channels. To investigate this, hydrogel patterning in 5 configurations with varying channel width and lumen diameter was tested. Firstly, gel was loaded to the first hydrogel channel and stopped immediately when it started to overflow. Following that, the gel was allowed to polymerize and the gel traveling distance was measured. While the gel was observed to overflow into the center lumen for channel width of 650 μm and 1 mm, the gel did not overflow until reaching the middle point of the channel for channel width of 1 mm (FIG. 30A). For channel width >1.5 mm, the gel can successfully travel from starting to ending point without overflowing to adjacent channels (FIG. 30A). Since the gel can reach middle point of the channel for channel width of 1 mm, it was proposed that gel can be loaded from one inlet to fill half of the channel length, followed which the remaining half can be loaded from the other inlet. As expected, chips with channel width of 1 mm can be robustly loaded using this “two-inlet-loading’ method (FIG. 30B). Upon patterning and polymerization of the 1^{st} lane of collagen (opaque-looking), the 2^{nd} lane of collagen (transparent-looking) was also successfully patterned using the “two-inlet-loading” method (FIG. 30B).

Next, the two chips (3/1/1 and 4/1/1) with the same channel width and different lumen diameter were used to find out whether lumen diameter affects the gel traveling distance. Collagen was loaded into the chip using the one-inlet loading method whereby gel loading was stopped immediately when overflowed. As a result, gel traveling distance for 4/1/1 is significantly longer than 3/1/1 (5.65±0.21 mm for 4/1/1 vs. 4.92±0.35 mm for 3/1/1) (FIGS. 31A and 31B).

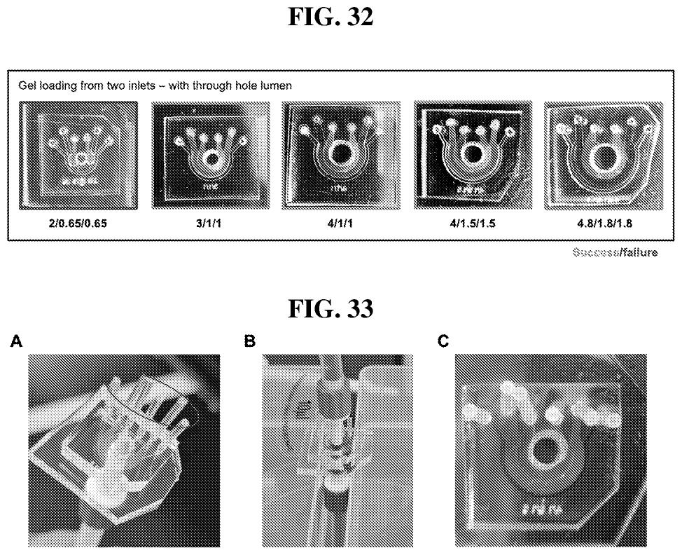

As described above, a significant advantage of the curved channel chip is the feasibility of incorporating fluid flow into the culture system by performing perfusion culture through the lumen. A through hole can be created by removing the PDMS of the lumen region to allow for connection with tubing and flow controllers. Upon removal of the PDMS at the lumen region, the stepped height conferring the CBV effect for the first hydrogel channel became bigger, hence the gel patterning property is expected to remain unchanged. Expectedly, the same results were achieved for all the designs with the through hole lumen (FIG. 32).

To test the feasibility of performing perfusion culture using this chip, the chip (4/1.5/1.5) was loaded with collagen for both channels, and the inlets were sealed with rods to prevent fluid leakage before perfusion (FIG. 33A). With continuous perfusion at 10 ml/min for 5 min, the gel was observed to remain intact without any displacement or breakage while effective diffusion of fluid from the center lumen to outer channels was established as evidenced by the distribution of red dye in the entire chip (FIG. 33C).

### Example 6: Commercial and Potential Applications

The spheroid-in-gel culture platform presented herein is poised to facilitate high throughput mechanistic studies and drug screening for cancer research or even other 3D cell models like stem cell organoids. The present platform offers compelling advantages over existing in vitro spheroid culture platforms owing to its ability to preserve the biological complexity needed for physiological relevancy while remaining scalable for high throughput studies. Therefore, it can be envisioned that it is of great interest for both fundamental scientific research (mechanistic studies) and translational research (high content drug screenings).

While the present disclosure has been particularly shown and described with reference to specific embodiments, it should be understood by those skilled in the art that various changes in form and detail may be made therein without departing from the spirit and scope of the present disclosure as defined by the appended claims. The scope of the present disclosure is thus indicated by the appended claims and all changes which come within the meaning and range of equivalency of the claims are therefore intended to be embraced.

1. A method of encapsulating a spheroid in a gel, the method comprising:
providing a frame comprising a base and an island protruding from the base;
depositing one or more suspensions on the island, wherein the one or more suspensions comprise different cells;
arranging the frame to have the one or more suspensions hang from the island in a direction which gravity acts to render growth of the spheroid;
depositing a gel on the spheroid with the spheroid resting on the island to have the gel encapsulate the spheroid; and
arranging the frame against a substrate with the base distally positioned from the substrate (i) to have the gel confined between the island and the substrate and (ii) to have the gel encapsulate the spheroid.

2. The method of claim 1, further comprising introducing one or more culture media to the gel after the gel encapsulates the spheroid.

3. The method of claim 1, wherein depositing the one or more suspensions comprise mixing the one or more suspensions with the gel prior to depositing the one or more suspensions.

4. The method of claim 1, wherein the one or more suspensions comprise a volume of at least 1 μL.

5. The method of claim 1, further comprising evaporating the one or more suspensions from the spheroid prior to depositing the gel.

6. The method of claim 1, wherein the gel comprises collagen, gelatin methacryloyl, and matrigel.

7. The method of claim 1, further comprising subjecting the gel to a temperature of 30 to 40° C. to render crosslinking within the gel for encapsulating the spheroid after arranging the frame against the substrate.

8. The method of claim 1, further comprising coating a layer of adhesive after crosslinking of the gel, wherein the adhesive comprises polydopamine, fibronectin, collagen, poly-L-lysine, or gelatin.

9. The method of claim 1, further comprising removing the frame from the substrate after the gel encapsulated the spheroid for retrieving the gel-encapsulated spheroid.

10. The method of claim 1, wherein the cells comprise human umbilical vein endothelial cells, human lung fibroblasts, or human breast cancer cells.

11. A device configured to render a spheroid encapsulated in a gel, the device comprising:
a frame comprising a base and an island protruding from the base; and
a substrate,
wherein the frame is arrangeable against the substrate with the base distally positioned from the substrate (i) to have the gel confined between the island and the substrate and (ii) to have the gel encapsulate the spheroid.

12. The device of claim 11,
wherein the frame comprises two supporting structures each configured at opposing edges of the base and extending therefrom, and
wherein the island is (i) configured between the two supporting structures, (ii) extends in the same direction as the two supporting structures from the base, and (iii) is vertically shorter than the two supporting structures.

13. The device of claim 12, wherein the frame further comprises two depressions each residing between one of the two supporting structures and the island.

14. The method of claim 12, wherein the two supporting structures extend at least 150 μm from the base.

15. The device of claim 11, wherein the island comprises:
one channel; or
more than one channel, wherein the more than one channel has the same or different depth.

16. The device of claim 11, wherein the island comprises at least one channel defined by multiple depressions, wherein each of the multiple depressions has a different depth.

17. The device of claim 11, wherein the island protrudes from the base at a height ranging from 10 μm to 500 μm.

18. The device of claim 11, wherein the island, when viewed from top down, comprises a circular shape, a three-sided shape, a four-sided shape, or a five-sided shape.

19. The device of claim 15, wherein the channel, or the more than one channel, is linear or curved.

20. The device of claim 16, wherein the island comprises at least one channel defined by multiple depressions, wherein each of the multiple depressions has a different depth, wherein the at least one channel extends horizontally across the island at a constant radius from a point in the island, and wherein the island comprises a lumen which extends vertically through the island.
